(19) United States
(12) Patent Application Publication
(10) Pub. No.: US 2024/0066172 A1
(43) Pub. Date: Feb. 29, 2024

(54) Volatilization Device with Novel Structure

(71) Applicant: GUANGDONG ILOVENUMBERONE CO.,LTD, Guangzhou (CN)

(72) Inventor: CHANGHUI LIU, Guangzhou (CN)

(21) Appl. No.: 17/896,100

(22) Filed: Aug. 26, 2022

(51) Int. Cl. A61L 9/12

(52) U.S. Cl. CPC A61L 9/12; A61L 2209/133

(57) ABSTRACT

The present utility model discloses a volatilization device with a novel structure, which includes a bottle body for storing aromatic liquid, a cover body movably connected to an opening of the bottle body, a base with a semi-open structure, and a diffusion sheet, wherein the diffusion sheet is disposed in the base, and a permeable membrane is disposed on the opening; and when in use, the opening is located in the base, and the permeable membrane is abutted against the diffusion sheet. The present utility model has the beneficial effects that the structure is simple, and unnecessary parts are discarded, so that the production cost is minimized; furthermore, an upside-down structure ensures that liquid can penetrate and volatilize completely; different diffusion rates can be achieved by using permeable membranes of different permeabilities; and in addition, decorations can be added in the bottle body to further improve the added value.

### TECHNICAL FIELD

The present utility model relates to the technical field of daily necessities, in particular to a volatilization device with a novel structure.

### BACKGROUND

Nowadays, using aromatics to provide fresh and fragrant indoor air is one of the choices in many places. At present, in terms of time, the commonly used aromatics are solid and liquid. However, both solid and liquid aromatics are loaded in containers and volatilized through cell bodies on the containers. However, these containers are all placed in a way of ‘standing upright’, and the problem is that even if some liquid aromatics have wicks, it is difficult to ensure that all aromatic liquid in the containers can be sucked out by the wicks, which will cause waste to a certain extent.

Although there are ‘upside-down’ aromatic liquid bottles on the market, these bottles are complex in structure and more in number of components, which increases the production cost in disguise; and on the other hand, some inconvenience will be also brought to consumers when they use the bottles (for example, Chinese patent aromatic bottle, publication number: CN 201734988), so it is necessary to re-optimize the upside-down aromatic bottles.

### SUMMARY

In view of the defects of the prior art, the present utility model aims to provide a volatilization device with a novel structure; due to arrangement of a permeable membrane, aromatic liquid in a bottle can be permeated at a certain rate and then transferred to a diffusion sheet, and is then volatilized to the surrounding environment through diffusion holes and/or diffusion cells on a base; and therefore, redundant structures are discarded to the maximum extent, and the product performance can be fully exerted while saving costs.

In order to achieve the above object, the present utility model adopts the following technical solution:

A volatilization device with a novel structure includes a bottle body for storing aromatic liquid, and a cover body movably connected to an opening of the bottle body, and further includes a base with a semi-open structure, and a diffusion sheet, where the diffusion sheet is disposed in the base, and a permeable membrane is disposed on the opening; and when in use, the opening is located in the base, and the permeable membrane is abutted against the diffusion sheet.

It should be pointed out that in addition to the aromatic liquid, the present utility model can also use deodorizing liquid, deworming liquid, sterilization liquid, etc.

It should be noted that a stepped structure is provided between the bottle body and the opening; and when the opening is located in the base, a peripheral edge of a surface of the stepped structure is movably connected to that of a top inner surface of the base.

It should be noted that a groove is formed in the peripheral edge of the surface of the stepped structure, and a flange corresponding to the groove is provided on the peripheral edge of the top inner surface of the base. Of course, the groove can be provided on the base, and the flange can be provided on the stepped structure, which does not affect the implementation of this solution.

It should be noted that the base is composed of a base body and a bottom plate movably connected to the base body.

It should be noted that peripheral edges of side walls of the base body are provided with diffusion holes and/or diffusion cells.

It should be pointed out that the permeable membrane of the present utility model can be sealed on the opening of the bottle body by hot pressing.

Further, the principle of the permeable membrane involved in the present utility model is basically the same as that of permeable membranes in the prior art.

Even further, the permeability of the permeable membrane involved in the present utility model can be set according to different needs when the permeable membrane is fabricated.

It should be further noted that the bottom plate in the base of the present utility model is provided with a protrusion portion, and the protrusion portion is used to support the diffusion sheet. The protrusion portion can be disposed in the center of the bottom plate, or there may be a plurality of protrusion portions, and the protrusion portions are evenly distributed on the bottom plate, which can also play the role of supporting the diffusion sheet; and furthermore, the structure can also increase the air circulation, thus facilitating the diffusion sheet to volatilize the liquid better.

The present utility model also includes another similar technical solution:

A volatilization device with a novel structure includes a bottle body for storing aromatic liquid, and a cover body movably connected to an opening of the bottle body, and further includes a base with a semi-open structure, and a diffusion sheet, where the diffusion sheet is disposed in the base, and a permeable membrane is disposed on the opening; and when in use, the opening is located in the base, and the permeable membrane is abutted against the diffusion sheet.

It should be noted that a support plate is disposed between the bottle body and the opening; and when the opening is located in the base, a peripheral edge of a bottom of the support plate is abutted against that of a top surface of the base.

It should be noted that the base is composed of a base body and a bottom plate movably connected to the base body.

It should be noted that peripheral edges of side walls of the base body are provided with diffusion holes and/or diffusion cells.

It should be pointed out that the connection manner of the base body and the bottom plate in this solution can be a buckle connection, plug-in connection, clamping connection, threaded connection, or the like in the prior art.

It should be pointed out that no matter which of the above solutions is adopted, in order to improve the ornamental performance of the present utility model, the bottle body can be made of transparent materials, and at the same time, decorations can be disposed in the bottle body, so that consumers not only clearly see the remaining amount of the aromatic liquid in the bottle body, but also see the decorations in the bottle body, thereby improving the value of the present utility model.

It should be further pointed out that when not in use, a surface of the permeable membrane of the present utility model is also provided with a layer of protective film, and the protective film is made of an air-impermeable material.

As another exemplary technical solution, the base of the present utility model may be an integrally-formed structure.

It should be further noted that a bottom of the base of the present utility model can also be provided with support legs.

The present utility model has the beneficial effects that the structure is simple, and unnecessary parts are discarded, so that the production cost is minimized; furthermore, an upside-down structure ensures that liquid can penetrate and volatilize completely; different diffusion rates can be achieved by using permeable membranes of different permeabilities; and in addition, decorations can be added in the bottle body to further improve the added value.

### BRIEF DESCRIPTION OF FIGURES

FIG. 1 is a structural schematic diagram of the present utility model;

FIG. 2 is a schematic diagram of another structure of the present utility model; and

FIG. 3 is a schematic diagram of still another structure of the present utility model.

### DETAILED DESCRIPTION

The technical solutions in the embodiments of the present utility model will be clearly and completely described below in conjunction with the accompanying drawings in the embodiments of the present utility model. Based on the embodiments of the present utility model, all other embodiments obtained by those of ordinary skill in the art without creative work fall within the protection scope of the present utility model.

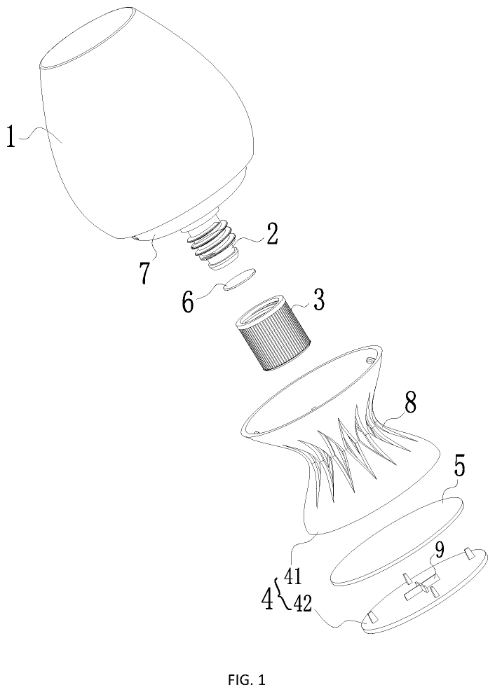

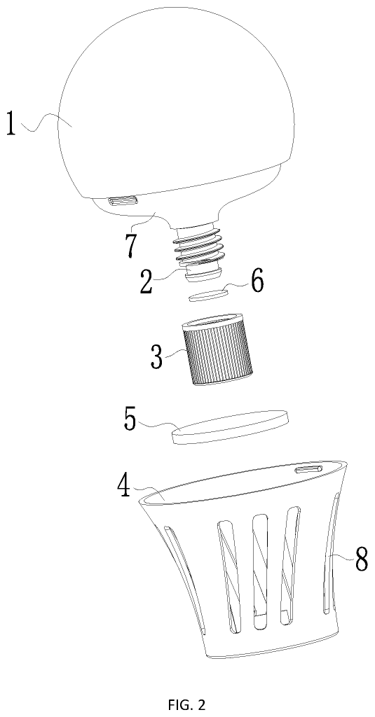

As shown in FIG. 1 and FIG. 2, the present utility model is a volatilization device with a novel structure, which includes a bottle body 1 for storing aromatic liquid, and a cover body 3 movably connected to an opening 2 of the bottle body 1, and further includes a base 4 with a semi-open structure, and a diffusion sheet 5, where the diffusion sheet 5 is disposed in the base 4, and a permeable membrane 6 is disposed on the opening 2; and when in use, the opening 2 is located in the base 4, and the permeable membrane 6 is abutted against the diffusion sheet 5.

It should be pointed out that in addition to the aromatic liquid, the present utility model can also use deodorizing liquid, deworming liquid, sterilization liquid, etc.

Further, as shown in FIG. 1 and FIG. 2, a stepped structure 7 is provided between the bottle body 1 and the opening 2; and when the opening 2 is located in the base 4, a peripheral edge of a surface of the stepped structure 7 is movably connected to that of a top inner surface of the base 4.

Further, according to the present utility model, a groove is formed in the peripheral edge of the surface of the stepped structure, and a flange corresponding to the groove is provided on the peripheral edge of the top inner surface of the base. Of course, the groove can be provided on the base, and the flange can be provided on the stepped structure, which does not affect the implementation of this solution.

Further, as shown in FIG. 1, the base 4 is composed of a base body 41 and a bottom plate 42 movably connected to the base body 41.

Further, as shown in FIG. 1 and FIG. 2, peripheral edges of side walls of the base body 41 are provided with diffusion holes and/or diffusion cells 8.

Further, the permeable membrane of the present utility model can be sealed on the opening of the bottle body by hot pressing; and of course, other sealing connection manners in the prior art can also be used.

Further, the principle of the permeable membrane involved in the present utility model is basically the same as that of permeable membranes in the prior art.

Even further, the permeability of the permeable membrane involved in the present utility model can be set according to different needs when the permeable membrane is fabricated.

Further, as shown in FIG. 1, the bottom plate 42 in the base 4 of the present utility model is provided with a protrusion portion 9, and the protrusion portion 9 is used to support the diffusion sheet 5. Even further, the protrusion portion can be disposed in the center of the bottom plate, or there may be a plurality of protrusion portions, and the protrusion portions are evenly distributed on the bottom plate, which can also play the role of supporting the diffusion sheet. Further, the structure can also increase the air circulation, thus facilitating the diffusion sheet to volatilize the liquid better.

The present utility model also includes another similar technical solution:

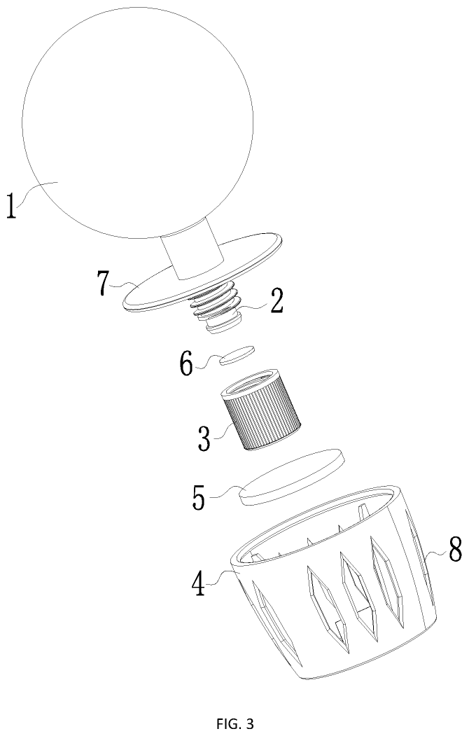

As shown in FIG. 3, the present utility model is a volatilization device with a novel structure, which includes a bottle body 1 for storing aromatic liquid, and a cover body 3 movably connected to an opening 2 of the bottle body 1, and further includes a base 4 with a semi-open structure, and a diffusion sheet 5, where the diffusion sheet 5 is disposed in the base 4, and a permeable membrane 6 is disposed on the opening 2; and when in use, the opening 2 is located in the base 4, and the permeable membrane 6 is abutted against the diffusion sheet 5.

Further, as shown in FIG. 3, a support plate 7 is disposed between the bottle body 1 and the opening 2; and when the opening 2 is located in the base 4, a peripheral edge of a bottom of the support plate 7 is abutted against that of a top surface of the base 4.

Further, according to the present utility model, the base is composed of a base body and a bottom plate movably connected to the base body.

Further, as shown in FIG. 3, according to the present utility model, peripheral edges of side walls of the base body are provided with diffusion holes and/or diffusion cells 8.

Further, the connection manner of the base body and the bottom plate in this solution can be a buckle connection, plug-in connection, clamping connection, threaded connection, or the like in the prior art.

It should be pointed out that no matter which of the above solutions is adopted, in order to improve the ornamental performance of the present utility model, the bottle body can be made of transparent materials, and at the same time, decorations can be disposed in the bottle body, so that consumers not only clearly see the remaining amount of the aromatic liquid in the bottle body, but also see the decorations in the bottle body, thereby improving the value of the present utility model.

The decorations mentioned above can be any kind of articles, such as ornaments, trinkets, paintings, toys, dolls, etc.

It should be further pointed out that a surface of the permeable membrane of the present utility model is also provided with a layer of protective film, and the protective film is made of an air-impermeable material, such as an aluminum film; and after the aluminum film is disposed, the structure can be further simplified, for example, the cover body can be removed.

It should be further noted that, in practice, the bottle body of the present utility model can be any shape in the prior art, such as a regular geometric shape or an irregular shape; and in addition, the diffusion sheet of the present utility model is consistent with diffusion sheets in the prior art, and can generally be made of sponge, fiber, cotton, paper and other materials.

As another exemplary technical solution, the base of the present utility model may be an integrally-formed structure.

It should be further noted that a bottom of the base of the present utility model can also be provided with support legs.

### Embodiment 1

When in use, a cover body is opened, and then a bottle body is put into a base in an upside-down way; at this time, a permeable membrane is in close face-contact with a diffusion sheet in the base, and under an action of gravity, aromatic liquid in the bottle body slowly seeps out through the permeable membrane and transfers onto the diffusion sheet; and when the diffusion sheet has the aromatic liquid, the aromatic liquid is volatilized to the surrounding environment through diffusion holes and/or diffusion cells reserved in the base. Consumers can not only see the remaining amount of the aromatic liquid in the bottle body, but also see the decorations in the bottle body.

### Embodiment 2

The overall structure of this Embodiment is basically the same as that of Embodiment 1. The difference is that in the structure of Embodiment 2, there is no cover body, but a protective film covers a permeable membrane, and can be removed when in use.

### Embodiment 3

The overall structure of this Embodiment is basically the same as that of Embodiment 1. The difference is that in the structure of Embodiment 3, there is a cover body, and a protective film covers a permeable membrane; and when in use, the cover body is removed, and then the protective film is removed.

For those skilled in the art, various corresponding changes and variations can be given according to the above technical solutions and concepts, and all these changes and variations should be included within the protection scope of the claims of the present utility model.

What is claimed is:

1. A volatilization device with a novel structure, comprising a bottle body for storing aromatic liquid, and a cover body movably connected to an opening of the bottle body, and further comprising a base with a semi-open structure, and a diffusion sheet, wherein the diffusion sheet is disposed in the base, and a permeable membrane is disposed on the opening; and when in use, the opening is located in the base, and the permeable membrane is abutted against the diffusion sheet.

2. The volatilization device with the novel structure according to claim 1, wherein a stepped structure is provided between the bottle body and the opening; and when the opening is located in the base, a peripheral edge of a surface of the stepped structure is movably connected to that of a top inner surface of the base.

3. The volatilization device with the novel structure according to claim 2, wherein a groove is formed in the peripheral edge of the surface of the stepped structure, and a flange corresponding to the groove is provided on the peripheral edge of the top inner surface of the base.

4. The volatilization device with the novel structure according to claim 1, wherein the base is composed of a base body and a bottom plate movably connected to the base body.

5. The volatilization device with the novel structure according to claim 4, wherein peripheral edges of side walls of the base body are provided with diffusion holes and/or diffusion cells.

6. A volatilization device with a novel structure, comprising a bottle body for storing aromatic liquid, and a cover body movably connected to an opening of the bottle body, and further comprising a base with a semi-open structure, and a diffusion sheet, wherein the diffusion sheet is disposed in the base, and a permeable membrane is disposed on the opening; and when in use, the opening is located in the base, and the permeable membrane is abutted against the diffusion sheet.

7. The volatilization device with the novel structure according to claim 6, wherein a support plate is disposed between the bottle body and the opening; and when the opening is located in the base, a peripheral edge of a bottom of the support plate is abutted against that of a top surface of the base.

8. The volatilization device with the novel structure according to claim 1, wherein the base is composed of a base body and a bottom plate movably connected to the base body.

9. The volatilization device with the novel structure according to claim 8, wherein peripheral edges of side walls of the base body are provided with diffusion holes and/or diffusion cells.

10. The volatilization device with the novel structure according to claim 2, wherein the base is composed of a base body and a bottom plate movably connected to the base body.

11. The volatilization device with the novel structure according to claim 3, wherein the base is composed of a base body and a bottom plate movably connected to the base body.
